(19) United States
(12) Patent Application Publication
(10) Pub. No.: US 2025/0339056 A1
(43) Pub. Date: Nov. 6, 2025

(54) METHOD FOR PROVIDING WALKING POSTURE CORRECTION INFORMATION AND DEVICE USING THE SAME

(71) Applicant: Jun beom KIM, Wonju-si (KR)

(72) Inventor: Jun beom KIM, Wonju-si (KR)

(21) Appl. No.: 19/265,347

(22) Filed: Jul. 10, 2025

Related U.S. Application Data
(63) Continuation of application No. PCT/KR2024/001813, filed on Feb. 7, 2024.

(30) Foreign Application Priority Data
Feb. 10, 2023 (KR) 10-2023-0018079

(51) Int. Cl. A61B 5/11; A61B 5/00

(52) U.S. Cl. CPC A61B 5/112; A61B 5/1116; A61B 5/1121; A61B 5/7271; A61B 2505/09

(57) ABSTRACT

A control method of a control device for correcting walking habits according to one embodiment includes obtaining a user's body information, obtaining an arm swing angle value and a sole landing angle value while the user walks, calculating a linked reference stride length value based on the arm swing angle value and the sole landing angle value, obtaining the user's heel width value, calculating a gap value between left and right inner sides of soles of both feet based on the user's heel width value, obtaining a forward hand direction value while the user walks, calculating a linked reference direction value based on the gap value between the left and right inner sides of the soles of both feet and the forward hand direction value, and providing walking habit correction information based on the linked reference stride length value and the linked reference direction value.

### CROSS-REFERENCE TO RELATED APPLICATIONS

This application is a continuation application of International Application No. PCT/KR2024/001813, filed on Feb. 7, 2024, which claims priority to Korean Patent Application No. 10-2023-0018079, filed on Feb. 10, 2023, the entire contents of which are hereby incorporated by references in its entirety.

### TECHNICAL FIELD

A method for providing walking posture correction information and a device using the same are disclosed.

### BACKGROUND ART

Posture and movement are interrelated. Good posture and good movement influence each other, but most people are not aware of the importance of posture and movement.

In particular, the balance between posture and movement is very important for adolescents during their growth period. Inappropriate posture and movement during adolescence can cause a physical imbalance and act as a negative factor in growth.

Currently, products for correction limited to certain parts of the body, such as spinal correction, joint correction, etc., are being released, but there is a limit in correcting the imbalance of body movement and posture that are interrelated. Accordingly, there is a need for the development of technology for effectively correcting a physical imbalance caused by users' inappropriate walking habits.

### DETAILED DESCRIPTION OF INVENTION

### [Technical Object]

Technical object is to analyze a user's walking habits and provide walking habit correction information to the user.

The technical object is not limited to the above-described object and other objects that are not described may be clearly understood by those skilled in the art from this specification and the accompanying drawings.

### Technical Solution

A control method of a control device for correcting walking habits according to one embodiment includes obtaining a user's body information, obtaining an arm swing angle value and a sole landing angle value while the user walks, calculating a reference stride length value linked to the user's arm swing angle and sole landing angle based on the arm swing angle value and the sole landing angle value, obtaining the user's heel width value, calculating a gap value between left and right inner sides of soles of both feet based on the user's heel width value, obtaining a forward hand direction value while the user walks, calculating a reference direction value linked to a gap between the inner sides of the soles of both feet based on the gap value between the left and right inner sides of the soles of both feet and the forward hand direction value, and providing walking habit correction information based on the linked reference stride length value and the linked reference direction value.

The technical solution is not limited to the above-described solution and other solutions that are not described may be clearly understood by those skilled in the art from this specification and the accompanying drawings.

### Advantageous Effects

According to the present application, by analyzing a user's walking habits and providing walking habit correction information to the user, it is possible to improve the user's walking habits, alleviate a postural imbalance of the user, and prevent and alleviate spinal-related diseases.

The effects of the present invention are not limited to the above-described effect and other effects that are not described may be clearly understood by those skilled in the art from this specification and the accompanying drawings.

### DESCRIPTION OF DRAWINGS

FIG. 1 is a diagram for describing a system according to one embodiment.

FIG. 2 is a diagram for describing a measuring device according to one embodiment.

FIG. 3 is a diagram for describing the arrangement of sensor units of a measuring device according to one embodiment.

FIG. 4 is a diagram for describing a control device according to one embodiment.

FIG. 5 is a diagram for describing a control method of a control device according to one embodiment.

FIG. 6 is a diagram for describing a walking posture according to a reference stride length value linked to an arm swing angle and sole landing angle according to one embodiment.

FIG. 7 is a diagram for describing a reference stride length value linked to an arm swing angle and sole landing angle according to one embodiment.

FIG. 8 is a diagram for describing a forward hand direction value according to one embodiment.

FIG. 9 is a diagram for describing a reference gap value between left and right inner sides of soles of both feet linked to a heel width according to one embodiment.

FIG. 10 is a diagram for describing a reference direction value linked to a gap between inner sides of soles of both feet and a hand direction.

FIG. 11 is a diagram for describing a control method of a control device according to another embodiment.

### BEST MODE

A control method of a control device for correcting walking habits according to one embodiment may include obtaining a user's body information, obtaining an arm swing angle value and a sole landing angle value while the user walks, calculating a reference stride length value linked to the user's arm swing angle and sole landing angle based on the arm swing angle value and the sole landing angle value, obtaining the user's heel width value, calculating a gap value between left and right inner sides of soles of both feet based on the user's heel width value, obtaining a forward hand direction value while the user walks, calculating a reference direction value linked to a gap between the inner sides of the soles of both feet based on the gap value between the left and right inner sides of the soles of both feet and the forward hand direction value, and providing walking habit correction information based on the linked reference stride length value and the linked reference direction value.

### Modes of the Invention

Since embodiments described in the present specification are intended to clearly explain the spirit of the present invention to those skilled in the art to which the present invention pertains, the present invention is not limited by the embodiments described herein and the scope of the present invention should be construed as including modifications, variations, equivalents, or substitutes that do not depart from the spirit of the present invention.

Although terms used herein are selected from among general terms that are currently and widely used in consideration of functions in the embodiments of the present invention, these may be changed according to intentions of those skilled in the art, precedents, or the advent of new technology. However, in a specific case, some terms may be arbitrary selected by the applicants. In this case, meanings thereof will be described in detail in a corresponding description of the embodiments of the present invention. Therefore, the terms used herein should be defined based on meanings of the terms and content of this entire specification, rather than simply the terms themselves.

The accompanying drawings of the present specification are for easy explanation of the present invention and the shapes illustrated in the drawings may be exaggerated and displayed as necessary to aid understanding of the present invention, and thus the present invention is not limited by the drawings.

In this specification, when it is determined that detailed descriptions of known configuration or function related to the present invention unnecessarily obscure the subject matter of the present invention, detailed descriptions thereof will be omitted as necessary.

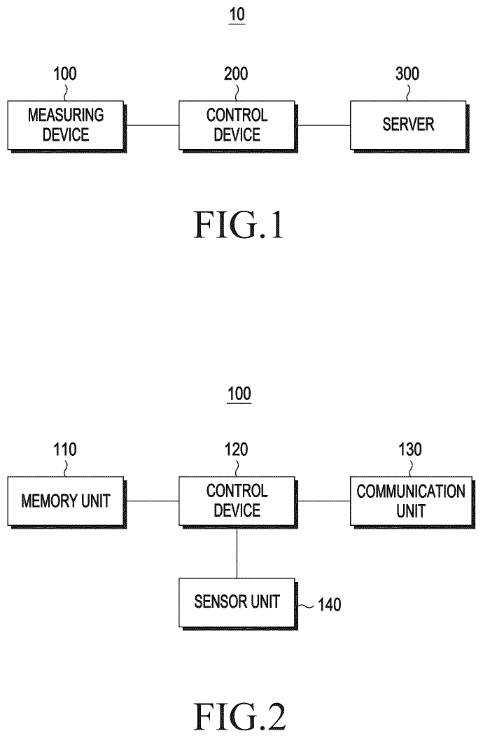

FIG. 1 is a diagram for describing a system according to one embodiment.

Referring to FIG. 1, a system 10 may include a measuring device 100, a control device 200, and a server 300.

The measuring device 100 may obtain the user's walking information. In one embodiment, the measuring device 100 in the form of a wearable device may be worn by the user. The measuring device 100 may include a sensor unit, and the sensor unit may be attached to the user's body so that the measuring device 100 may obtain the user's walking information and/or body information through the sensor unit. For example, the measuring device 100 may measure the user's arm swing angle, sole landing angle, stride length, forward hand direction, gap between inner sides of soles of both feet, etc., while the user walks. The measuring device 100 will be described in detail with reference to FIG. 2.

Further, the control device 200 may calculate reference walking information according to the user's body based on the walking information and/or body information obtained from the measuring device 100. Further, the control device 200 may compare the walking information obtained from the measuring device 100 with the reference walking information to determine the user's walking habits. When it is determined that the user's walking habits are inappropriate, the control device 200 may provide walking habit correction information to the user. Further, the control device 200 may communicate with the server 300. The control device 200 may provide various types of information, such as the walking information obtained from the measuring device 100, the reference walking information, information on the determined walking habits, the walking habit correction information provided to the user, etc., to the server 300. In one embodiment, the control device 200 and the measuring device 100 may be implemented physically independently, or may be implemented as a single device. Further, in one embodiment, the control device 200 may be implemented in the form of a mobile device such as a mobile, a smartphone, a tablet, or a personal computer (PC), or a fixed device such as a computer or a server.

The server 300 may communicate with the control device 200 and/or the measuring device 100 and monitor the control device 200. The server 300 may record and manage information provided from the control device 200 and/or the measuring device 100. Further, in some embodiments, the server 300 may calculate the reference walking information in a manner similar to the control device 200, or compare the obtained walking information with the reference walking information to determine the user's walking habits, and when it is determined that the user's walking habits are inappropriate, provide the walking habit correction information to the user. Further, in some embodiments, the server 300 may remotely control the control device 200. For example, the server 300 may transmit a control signal for controlling the control device 200 to the control device 200 and receive information on a result of the operation according to the control signal from the control device 200.

FIG. 2 is a diagram for describing the measuring device according to one embodiment.

Referring to FIG. 2, the measuring device 100 may include a memory unit 110, a control unit 120, a communication unit 130, and sensor unit 140.

The control unit 120 may control the overall operation of the measuring device 100. For example, the control unit 120 may process information measured by the sensor unit 140 to obtain walking information and/or body information.

The control unit 120 may include one or more of a central processing unit (CPU), a graphics processing unit (GPU), one or more microprocessors, and other electronic parts capable of processing input data according to predetermined logic.

Further, the control unit 120 may be implemented in software, hardware, and a combination thereof. For example, in hardware, the control unit 120 may be implemented with a field-programmable gate array (FPGA), an application specific integrated circuit (ASIC), a semiconductor chip, and other various types of electronic circuits. In addition, for example, in software, the control unit 120 may be implemented with a logic program or various computer languages that are executed according to the hardware described above.

Further, the memory unit 110 may store instructions for controlling the control unit 120, and the control unit 120 may perform operations based on the instructions stored in the memory unit 110. Further, the memory unit 110 may store information necessary for the operation of the control unit 120.

Further, the memory unit 110 may include at least one type of storage medium among a flash memory type memory, a hard disk type memory, a multimedia card micro type memory, a card type memory (e.g., a secure digital (SD) card memory or an xD card memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. In addition, the memory unit 110 may temporarily, permanently, or semi-permanently store information, and may be provided as a built-in or removable type.

Further, the communication unit 130 may communicate with the control device 200. Further, in some embodiments, the communication unit 130 may communicate with the server 300 and/or another external device.

The communication unit 130 may mainly perform communication according to wireless communication standards, but may also include wired or wireless modules that transmit data through various communication standards, including Bluetooth Low Energy (BLE), Bluetooth, a wireless local area network (WLAN), Wireless Fidelity (Wi-Fi), Wi-Fi Direct, near-field communication (NFC), infrared data association (IrDA), ultra-wide band (UWB), ZigBee, third generation (3G), fourth generation (4G), or fifth generation (5G), and other mobile communication modules. Further, the communication unit 130 may include a short-range wireless module that supports NFC, radio-frequency identification (RFID), etc. Further, the communication unit 130 may include a wireless module that supports a wireless mesh network (WMN). For example, a predetermined low-power wireless communication (e.g., a wireless personal area network (WPAN)) method may be used in the WMN.

Further, the sensor unit 140 may measure the movement of each part of the user's body according to walking.

In one embodiment, the sensor unit 140 may be placed on various parts of the user's body. For example, the sensor unit 140 may be placed on the user's body in the form of a wearable device or may be directly attached to the user's body.

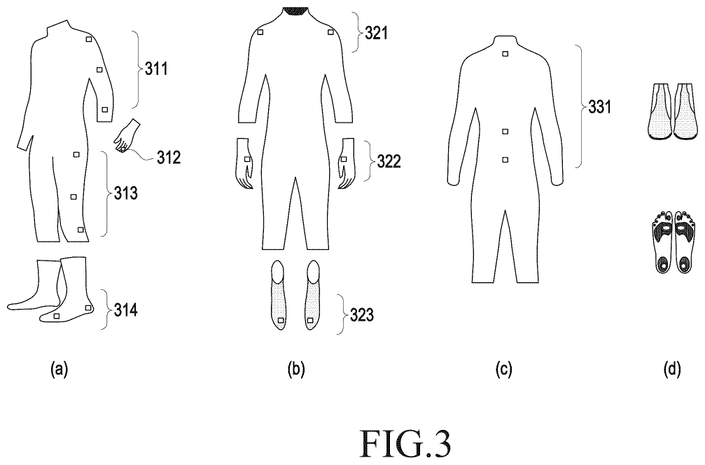

The arrangement of the sensor unit 140 will be described with reference to FIG. 3, and FIG. 3 is a set of diagrams for describing the arrangement of the sensor units of the measuring device according to one embodiment.

In (a) of FIG. 3, the sensor unit 140 may measure front and rear angles of the user's body. For example, the sensor unit 140 may be placed on the user's arm as shown with reference numeral 311 to measure the user's arm swing angle. Further, the sensor unit 140 may be placed on the user's fingertip and/or the back of the user's hand as shown with reference numeral 312 to measure the user's arm swing angle and/or forward hand direction. Further, the sensor unit 140 may be placed on the user's leg as shown with reference numeral 313 to measure forward and rearward angles of the user's leg. Further, the sensor unit 140 may be placed on the user's foot as shown with reference numeral 314 to measure a sole landing angle.

In (b) to (d) of FIG. 3, the sensor unit 140 may measure left and right directions and the balance of the user's body. For example, the sensor unit 140 may be placed on the user's shoulders as shown with reference numeral 321 to measure shoulder balance. Further, the sensor unit 140 may be placed on sides of the user's hands as shown with reference numeral 322 to measure directions of the user's hands. Further, the sensor unit 140 may be placed on the user's toes as shown with reference numeral 323 to measure directions of the user's toes. Further, the sensor unit 140 may be placed on the user's spine (7^{th} cervical vertebra, 12^{th} thoracic vertebra, or 5th lumbar vertebra) as shown with reference numeral 331 to measure the balance of the spine. Further, the sensor unit 140 may be placed on tips of the user's toes or the user's soles to measure directions of the user's soles.

Further, in some embodiments, the sensor unit 140 may be placed on each body part as described above or a plurality of sensor unit 140 may be placed thereon.

Further, in some embodiments, the sensor unit 140 may be composed of various sensors such as an acceleration sensor, an angular velocity sensor, a gyro sensor, an orientation sensor, an angle sensor, a pressure sensor, a three-axis or more inertial sensor, a carbon nanotube (CNT) strain sensor, etc.

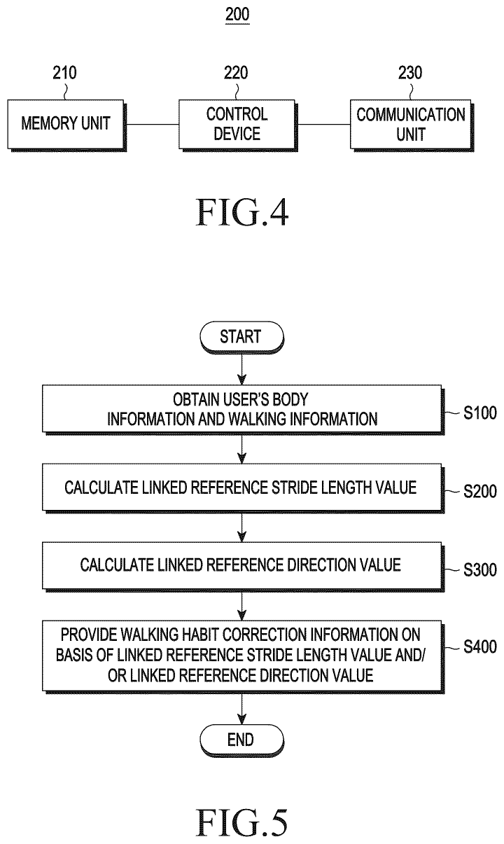

FIG. 4 is a diagram for describing the control device according to one embodiment.

Referring to FIG. 4, the control device 200 may include a memory unit 210, a control unit 220, and a communication unit 230.

The control unit 220 may control the overall operation of the control device 200. For example, the control unit 220 may calculate reference walking information based on walking information and/or body information obtained from the measuring device 100 and determine user's walking habits based on the reference walking information. Further, the control unit 220 may provide walking habit correction information to the user according to the user's walking habits.

The control unit 220 may include one or more of a CPU, a GPU, one or more microprocessors, and other electronic parts capable of processing input data according to predetermined logic.

Further, the control unit 220 may be implemented in software, hardware, and a combination thereof. For example, in hardware, the control unit 220 may be implemented with an FPGA, an ASIC, a semiconductor chip, and other various types of electronic circuits. In addition, for example, in software, the control unit 220 may be implemented with a logic program or various computer languages that are executed according to the hardware described above.

Further, the memory unit 210 may store instructions for controlling the control unit 220, and the control unit 220 may perform operations based on the instructions stored in the memory unit 210. Further, the memory unit 210 may store information necessary for the operation of the control unit 220.

Further, the memory unit 210 may include at least one type of storage medium among a flash memory type memory, a hard disk type memory, a multimedia card micro type memory, a card type memory (e.g., an SD card memory or an xD card memory, etc.), a RAM, an SRAM, a ROM, an EEPROM, a PROM, a magnetic memory, a magnetic disk, and an optical disk. In addition, the memory unit 210 may temporarily, permanently, or semi-permanently store information, and may be provided as a built-in or removable type.

Further, the communication unit 230 may communicate with the measuring device 100. Further, in some embodiments, the communication unit 230 may communicate with the server 300 and/or another external device.

The communication unit 230 may mainly perform communication according to wireless communication standards but may also include wired or wireless modules that transmit data through various communication standards, including BLE, Bluetooth, a WLAN, Wi-Fi, Wi-Fi Direct, NFC, IrDA, UWB, ZigBee, 3G, 4G, or 5G, and other mobile communication modules. Further, the communication unit 230 may include a short-range wireless module that supports NFC, RFID, etc. Further, the communication unit 230 may include a wireless module that supports a WMN. For example, a predetermined low-power wireless communication (e.g., a WPAN) method may be used in the WMN.

FIG. 5 is a diagram for describing a control method of a control device according to one embodiment.

Referring to FIG. 5, the control method of the control device may include operation S100 of obtaining a user's body information and walking information, operation S200 of calculating a linked reference stride length value, operation S300 of calculating a linked reference direction value, and operation S400 of providing walking habit correction information based on the linked reference stride length value and/or the linked reference direction value.

In operation S100, the control device may obtain the user's body information and walking information. For example, the control device may obtain information, such as age information, gender information, height information, heel width information, etc. of the user, as the user's body information. As an example, the control device may obtain the user's body information from a measuring device. As another example, the control device may further include an input unit (not illustrated), and may obtain the user's body information through the input unit (not illustrated). As still another example, the control device may obtain the user's body information through communication with an external device (e.g., a mobile device).

Further, the control device may obtain the user's walking habit information, such as the user's arm swing angle, sole landing angle, linked stride length, forward hand direction, gap between inner sides of soles of both feet, heel width value, linked direction, etc., while the user walks. As an example, the user's walking information may be obtained from the control device. As another example, the control device may obtain the user's walking information through the input unit (not illustrated) and obtain the user's body information through communication with an external device (e.g., a mobile device).

Further, in operation S200, the control device may calculate the linked reference stride length value.

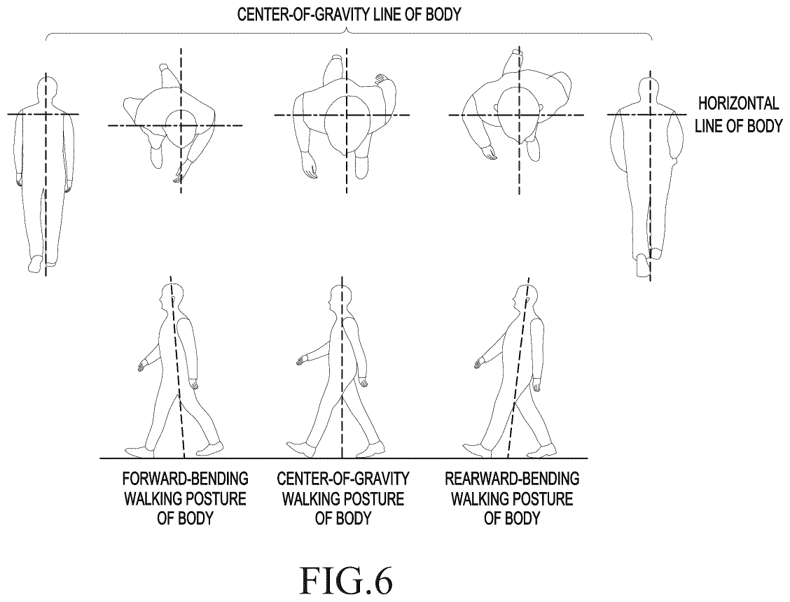

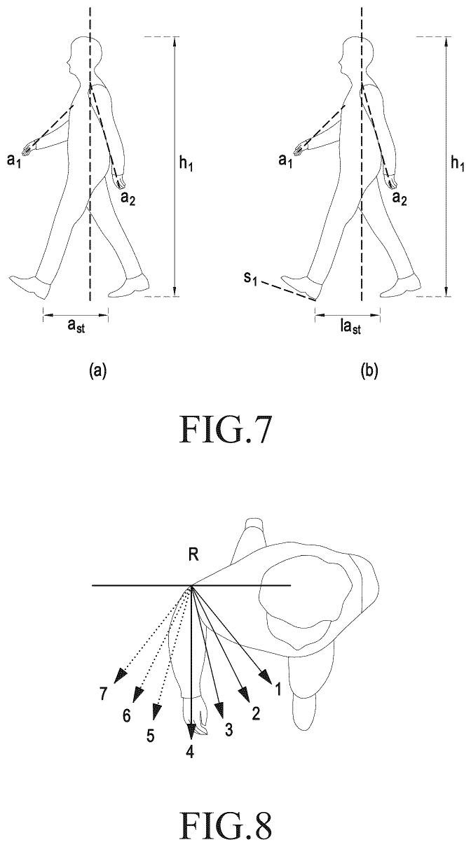

In one embodiment, the control device may calculate a reference stride length value linked to the arm swing angle based on the user's height value and arm swing angle value. Further, the control device may calculate the linked reference stride length value based on an arm swing angle stride length value, an arm swing angle value and a sole landing angle value. This will be described with reference to FIGS. 6 and 7.

First, for convenience of description, terms and formulas will be described. Table 1 below is an explanation of the official terms, initials, and subscripts of a linked stride length and linked direction for determining the appropriateness or inappropriateness of body position on the front-rear center-of-gravity line and on left-right horizontal line of the present invention.

TABLE 1
Official terms

| Content | | | Initials | Subscripts |
|---|---|---|---|---|
| Linked | Height | height | H | — |
| stride | Reference stride length value linked to | arm step | AS | aₛₜ |
| length | arm swing angle | | | |
| | Reference stride length value linked to | linked arm step | LAS | laₛₜ |
| | arm swing angle and sole landing | | | |
| | angle | | | |
| Linked | Forward hand direction value | hand direction value | HDV | h_{dv} |
| direction | Forward hand direction calculation | hand direction | HDCV | h_{dcv} |
| | value | calculation value | | |
| | Medial forward hand direction | medial hand direction | MHDCV | — |
| | calculation value | calculation value | | |
| | External forward hand direction | external hand direction | EHDCV | |
| | calculation value | calculation value | | |
| | Heel width value | sole heel area | SHA | sₕₐ |
| | Reference gap value between left and | heel space | HS | hₛₚ |
| | right inner sides of soles of both feet | | | |
| | linked to heel width | | | |
| | Gap between left and right inner sides | inside heel space | IHS | — |
| | of soles of both feet in inward direction | | | |
| | Gap between left and right inner sides | outside heel space | OHS | |
| | of soles of both feet in outward | | | |
| | direction | | | |
| | Reference direction value linked to gap | linked hand heel | LHH | lₕₕ |
| | between inner sides of soles of both | | | |
| | feet and forward hand direction | | | |

Further, Table 2 provides an explanation of the subscript contents, formulas, and substitution calculation formulas for the linked stride length and linked direction, which are used to determine whether the body is appropriately positioned on the anterior-posterior center-of-gravity line and the left-fight horizontal line of the present invention.

TABLE 2

| | Subscript contents | Formulas |
|---|---|---|
| Linked | aₛₜ | aₛₜ = h₁*h₂*(a₁ + a₂)/a₃ |
| stride | (arm step) | |
| formulas | laₛₜ | laₛₜ = h₁*h₂*((a₁ + a₂)/a₃)*(s₁/s₂) |
| | (Linked arm step) | |
| Linked | h_{dv} | h_{dv} = h_{dcv}*h₃ |
| direction | (hand direction value) | |
| formulas | hₛₚ | hₛₚ = sₕₐ*h₄ |
| | (heel space) | |
| | lₕₕ | lₕₕ = h_{dcv}*h₃*sₕₐ*h₄ |
| | (Linked hand heel) | |

First, referring to FIG. 6, FIG. 6 is a diagram for describing a walking posture according to a reference stride length value linked to an arm swing angle and sole landing angle according to one embodiment.

In FIG. 6, a dotted line represents the center-of-gravity line of the body front and rear, and a dot-dash line represents a horizontal line of the body left and right. In one embodiment, an appropriate reference stride length may be determined based on the arm swing angle and/or the sole landing angle while the user walks. While the user walks with a corresponding reference stride length, the user's body front-rear center-of-gravity line and the body left-right horizontal line may not be tilted, and thus the walking posture may be correct. For example, while the user walks with the corresponding reference stride length, the user may walk correctly with the center-of-gravity walking posture of the body.

However, while the user walks with a stride length that is too short or too long compared to the corresponding reference stride length, the body front-rear center-of-gravity line and the left-right horizontal line may be tilted, and thus the walking posture may be inappropriate. For example, while the user walks with a stride length that is too short or too long compared to the corresponding reference stride length, the user may walk with an inappropriate walking posture such as a forward-bending walking posture of the body or a rearward-bending walking posture of the body, which may result in an imbalance in the body.

Accordingly, the control device may calculate the linked reference stride length value based on the user's arm swing angle and/or sole landing angle and may determine whether the user's walking habits are correct based on the linked reference stride length value. This will be described with reference to FIG. 7.

FIG. 7 is a set of diagrams for describing a reference stride length value linked to an arm swing angle and sole landing angle according to one embodiment.

Referring to FIG. 7, (a) is a diagram illustrating a reference stride length value linked to an arm swing angle, and (b) is a diagram for describing the reference stride length value linked to the arm swing angle and sole landing angle.

In (a) of FIG. 7, in the arm swing angle-linked reference stride length value aₛₜ, a₁ may denote a maximum forward arm angle value among arm swing angles, and a₂ may denote a maximum rearward arm angle value among the arm swing angles. Further, h₁ may denote the user's height value.

For healthy walking habits, an appropriate stride length may be required according to the arm swing angle. When the stride length is too narrow or too wide compared to the arm swing angle, the body balance may be lost. Accordingly, the control device may calculate the arm swing angle-linked reference stride length value aₛₜ to determine whether the user's walking habits are appropriate.

In one embodiment, the control device may calculate the arm swing angle-linked reference stride length value aₛₜ using Table 2 and Equation 1 below.

$${a}_{st} = {h}_{1} \times {h}_{2} \times \frac{{a}_{1} + {a}_{2}}{{a}_{3}} \qquad [ Equation 1 ]$$

Here, h₁ denotes the user's height value, h₂ denotes a reference value of a stride length range of a center-of-gravity walking posture, a₁ denotes the maximum forward arm angle value, a₂ denotes the maximum rearward arm angle value, and a₃ denotes a reference value of an arm swing angle range of the center-of-gravity walking posture.

When the maximum forward arm angle value is a₁ degrees and the maximum rearward arm angle value is a₂ degrees, the arm swing angle-linked reference stride length value aₛₜ may be calculated based on the user's height value h₁, the reference value h₂ of the stride length range of the center-of-gravity walking posture, and the reference value a₃ of the arm swing angle range. In one embodiment, the reference value of the arm swing angle range of the center-of-gravity walking posture and the reference value of the stride length range of the center-of-gravity walking posture may be predetermined. Further, in one embodiment, the maximum forward arm angle value and the maximum rearward arm angle value may also be predetermined. In this case, the arm swing angle-linked reference stride length value may vary according to the user's height value.

Further, in one embodiment, the control device may pre-generate and store a lookup table as shown in Table 3 below based on Equation 1 described above. In this case, the control device may pre-store the lookup table and extract the arm swing angle-linked reference stride length value aₛₜ based on the user's arm swing angle and height value. Of course, the control device may generate a lookup table in more detail than that in Table 3 below.

Further, in the above-described example, when the user's height value is 180, the maximum forward arm angle value is 30 degrees, and the maximum rearward arm angle value is 20 degrees, as shown in Table 3 below, the arm swing angle-linked reference stride length value may be aₛₜ 1819. In Table 3, aₛₜ 1111 to aₛₜ 1825 are pre-calculated numbers, and may be changed according to the reference value of the arm swing angle range of the center-of-gravity walking posture and the reference value of the stride length range of the center-of-gravity walking posture.

TABLE 3

| | Maximum forward | | | |
|---|---|---|---|---|
| | arm angle/ | Height value h₁ | | |
| | maximum | 110 | . . . | 180 |
| | rearward arm | Arm swing forward-rearward angle-linked | | |
| | angle a₁/a₂ | reference stride length value aₛₜ | | |
| 1 | 05°/05° | aₛₜ1111 | . . . | aₛₜ1811 |
| 2 | 10°/05° | aₛₜ1112 | . . . | aₛₜ1812 |
| 3 | 10°/10° | aₛₜ1113 | . . . | aₛₜ1813 |
| 4 | 15°/10° | aₛₜ1114 | . . . | aₛₜ1814 |
| 5 | 20°/10° | aₛₜ1115 | . . . | aₛₜ1815 |
| 6 | 20°/15° | aₛₜ1116 | . . . | aₛₜ1816 |
| 7 | 25°/15° | aₛₜ1117 | . . . | aₛₜ1817 |
| 8 | 25°/20° | aₛₜ1118 | . . . | aₛₜ1818 |
| 9 | 30°/20° | aₛₜ1119 | . . . | aₛₜ1819 |
| 10 | 35°/20° | aₛₜ1120 | . . . | aₛₜ1820 |
| 11 | 35°/25° | aₛₜ1121 | . . . | aₛₜ1821 |
| 12 | 40°/25° | aₛₜ1122 | . . . | aₛₜ1822 |
| 13 | 40°/30° | aₛₜ1123 | . . . | aₛₜ1823 |
| 14 | 45°/30° | aₛₜ1124 | . . . | aₛₜ1824 |
| 15 | 45°/35° | aₛₜ1125 | . . . | aₛₜ1825 |

Further, in one embodiment, the control device may determine whether the user's maximum forward arm angle and maximum rearward arm angle are within a predetermined range from a predetermined maximum forward arm angle and maximum rearward arm angle to a predetermined angle. For example, when the predetermined maximum forward arm angle is predetermined to be a₁ degrees and the predetermined maximum rearward arm angle is predetermined to be a₂ degrees, information on the user's maximum forward arm angle and maximum rearward arm angle may be obtained. When the user's maximum forward arm angle and maximum rearward arm angle are not within the predetermined range of the arm swing angle from the predetermined maximum forward arm angle, a₁ degrees, and maximum rearward arm angle, a₂ degrees, information for correcting the user's maximum forward arm angle and maximum rearward arm angle may be provided. For example, the control device may provide information on a difference value between the user's maximum forward arm angle and maximum rearward arm angle and the predetermined maximum forward arm angle, a₁ degrees, and maximum rearward arm angle, a₂ degrees.

Further, in one embodiment, the control device may determine whether the user's stride length value is within a range from the arm swing angle-linked reference stride length value to a predetermined value. When the user's stride length value is not within the range from the arm swing angle-linked reference stride length value to a predetermined value, information for correcting the user's stride length value may be provided. For example, the control device may provide information on a difference value between the user's stride length value and the arm swing angle-linked reference stride length value.

Further, in one embodiment, the sole landing angle may also be considered in calculating the linked reference stride length value. This is because, while the user walks with a stride length that does not correspond to the sole landing angle, the user's body balance may be disrupted due to a forward-bending walking posture of the body or a rearward-bending walking posture of the body. Accordingly, the control device may calculate a reference stride length value laₛₜ linked to the arm swing angle and sole landing angle to determine whether the user's walking habits are appropriate.

In one embodiment, the control device may calculate the reference stride length value laₛₜ linked to the arm swing angle and sole landing angle using Equation 2 below.

$${la}_{st} = {h}_{1} \times {h}_{2} \times \frac{{a}_{1} + {a}_{2}}{{a}_{3}} \times \frac{{s}_{1}}{{s}_{2}} \qquad [ Equation 2 ]$$

Here, h₁ denotes the user's height value, h₂ denotes the reference value of the stride length range of the center-of-gravity walking posture, a₁ denotes the maximum forward arm angle value, a₂ denotes the maximum rearward arm angle value, and a₃ denotes the reference value of the arm swing angle range of the center-of-gravity walking posture. Further, s₁ denotes the user's sole landing angle, and s₂ denotes the reference value of the sole landing angle range of the center-of-gravity walking posture. In one embodiment, the reference value of the sole landing angle range of the center-of-gravity walking posture may be predetermined based on a predetermined sole landing angle.

For example, when a predetermined sole landing angle value is 40 degrees, a sole landing angle correction value may be 40 degrees. Of course, the above-described sole landing angle correction value may be a value other than this. As in Equations 1 and 2, the reference stride length value laₛₜ linked to the arm swing angle and sole landing angle may be based on the arm swing angle-linked reference stride length value aₛₜ. Accordingly, the reference stride length value laₛₜ linked to the arm swing angle and sole landing angle may be based on the arm swing angle-linked reference stride length value aₛₜ, the user's sole landing angle s₁, and the reference value s₂ of the sole landing angle range of the center-of-gravity walking posture.

Further, in one embodiment, the control device may pre-generate and store a lookup table as shown in Table 4 below based on Equation 2 described above. In this case, the control device may pre-store the lookup table and extract the reference stride length value laₛₜ linked to the arm swing angle and sole landing angle based on the user's height value and the user's arm swing angle-linked reference stride length value and sole landing angle. Of course, the control device may generate a lookup table in more detail than Table 4 below.

Further, in the above-described example, when the user's height value is 180, the arm swing angle-linked reference stride length value is aₛₜ 1819, and the sole landing angle is 35 degrees, as shown in Table 4 below, the reference stride length value linked to the arm swing angle and sole landing angle may be laₛₜ 1819. In Table 4, laₛₜ1114 to laₛₜ1821 are pre-calculated numbers, and may be changed according to the reference value of the arm swing angle range of the center-of-gravity walking posture, the reference value of the stride length ange of the center-of-gravity walking posture, and the reference value of the sole landing angle range of the center-of-gravity walking posture. Further, for example, in Table 4 below, although it is assumed that the maximum forward arm angle value a₁ and the maximum rearward arm angle value a₂ are 30 degrees and 20 degrees, respectively, the present invention is not limited thereto, and various lookup tables for the maximum forward arm angle value a₁ and the maximum rearward arm angle value a₂ may also be generated.

TABLE 4

| | | Height value h₁ | | |
|---|---|---|---|---|
| | | 110 | . . . | 180 |
| | | Arm swing forward-rearward angle-linked reference | | |
| | Sole | stride length value aₛₜ | | |
| | landing | aₛₜ1119 | | aₛₜ1819 |
| | angle | Arm swing angle and sole landing angle-linked | | |
| | s₁ | reference stride length value last | | |
| 1 | 10° | laₛₜ1114 | . . . | laₛₜ1814 |
| 2 | 15° | laₛₜ1115 | . . . | laₛₜ1815 |
| 3 | 20° | laₛₜ1116 | . . . | laₛₜ1816 |
| 4 | 25° | laₛₜ1117 | . . . | laₛₜ1817 |
| 5 | 30° | laₛₜ1118 | . . . | laₛₜ1818 |
| 6 | 35° | laₛₜ1119 | . . . | laₛₜ1819 |
| 7 | 40° | laₛₜ1120 | . . . | laₛₜ1820 |
| 8 | 45° | laₛₜ1121 | . . . | laₛₜ1821 |

Further, in one embodiment, the control device may determine whether the user's sole landing angle is within a range from the predetermined sole landing angle to a predetermined angle. When the user's sole landing angle is not within the range from the predetermined sole landing angle to the predetermined angle, information for correcting the user's sole landing angle may be provided. For example, when the predetermined sole landing angle is predetermined to be s₁, the control device may provide information on a difference value between the user's sole landing angle and the predetermined sole landing angle s₁.

Further, in one embodiment, the control device may determine whether the user's stride length value is within a range from the reference stride length value laₛₜ linked to the arm swing angle and sole landing angle to a predetermined value. When the user's stride length value is not within the range from the reference stride length value laₛₜ linked to the arm swing angle and sole landing angle to the predetermined value, information for correcting the user's stride length value may be provided. For example, the control device may provide information on a difference value between the user's stride length value and the reference stride length value laₛₜ linked to the arm swing angle and sole landing angle.

Referring to FIG. 5 again, in operation S300, the control device may obtain a forward hand direction value. Specifically, the control device may obtain a forward hand direction measurement value from the measuring device. Further, the control device may obtain the forward hand direction value from the forward hand direction measurement value. This will be described with reference to FIG. 8 and Table 5 below.

TABLE 5
Forward hand direction range (units: degrees)

| | | | | | | |
|---|---|---|---|---|---|---|
| −24~−34 | −13~−23 | −2~−12 | −1~1 | 2~12 | 13~23 | 24~34 |

Forward hand direction calculation values h_{dcv}

| | | | | | | |
|---|---|---|---|---|---|---|
| 1 | 2 | 3 | 4 | 5 | 6 | 7 |

Forward hand direction values h_{dv}

| | | | | | | |
|---|---|---|---|---|---|---|
| h_{dv}1025 | h_{dv}1050 | h_{dv}1075 | h_{dv}1111 | h_{dv}1125 | h_{dv}1150 | h_{dv}1175 |

FIG. 8 is a diagram for describing a forward hand direction value according to one embodiment.

Referring to FIG. 8, numbers 1 to 7 may represent forward hand direction calculation values h_{dvc} of Table 5 above. The control device may compare the user's forward hand direction measurement values with the forward hand direction ranges of Table 5 to obtain the forward hand direction calculation values h_{dcv}. Here, the user's forward hand direction measurement values may be measured to be 0 degrees when a forward hand direction is vertical with respect to the user's body left-right horizontal line R, to be negative angles (1 to 3) when the forward hand direction is directed toward the user's torso, and to be positive angles (5 to 7) when the forward hand direction is directed outward. For example, when the user's forward hand direction measurement value is 0 degrees, 4 may be obtained as the forward hand direction calculation value h_{dcv} when the user's forward hand direction measurement value is 15 degrees, 6 may be obtained as the forward hand direction calculation value h_{dvc} and when the user's forward hand direction measurement value is −25 degrees, 1 may be obtained as the forward hand direction calculation value h_{dcv}.

Further, the control device may calculate a forward hand direction value h_{dv} using Equation 3 below.

$${h}_{dv} = {h}_{dev} \times {h}_{3} \qquad [ Equation 3 ]$$

Here, h_{dv} may denote the forward hand direction value, h_{dvc} may denote the forward hand direction calculation value, and h3 may denote a reference value of a forward hand direction range of a center-of-gravity walking posture.

Further, in one embodiment, the control device may set the forward hand direction calculation value h_{dcv}, 4, as the reference forward hand direction value h_{dv}. This is because, when the forward hand direction value h_{dv} is vertical with respect to the body left-right horizontal line R during walking, an appropriate walking posture may be maintained.

Further, in one embodiment, the control device may determine whether the user's forward hand direction value is within a range from a predetermined forward hand direction value (e.g., a forward hand direction value, h_{dv} 1111, of Table 5) to a predetermined value. When the user's forward hand direction value is not within the range from the predetermined forward hand direction value to the predetermined value, information for correcting the user's forward hand direction value may be provided. For example, the control device may provide information on a difference value between the user's forward hand direction value and the predetermined forward hand direction value or information on a difference value between the user's forward hand direction value and a forward hand direction range corresponding to the predetermined forward hand direction value.

Referring to FIG. 5 again, in operation S300, the control device may calculate the linked reference direction value. In one embodiment, the control device may calculate a reference gap value between inner sides of soles of both feet linked to the heel width based on the user's heel width value and obtain the above-described forward hand direction value. Further, the control device may calculate the reference direction value linked to the gap between the inner sides of the soles of both feet and the forward hand direction.

Specifically, the control device may calculate a reference gap value between left and right inner sides of the soles of both feet linked to the heel width based on the user's heel width value. More specifically, the control device may obtain the user's heel width value. In one embodiment, the user's heel width value may be obtained in operation S100. The control device may obtain the user's heel width value at a specific location on the user's sole. For example, the control device may obtain the user's heel width value at a specific location from the tip of the user's heel. Further, the control device may obtain the user's heel width value from the measuring device. In this case, the sensor unit of the measuring device may be placed on the user's heel. Further, the control device may obtain the user's heel width value from an input unit (not illustrated) or an external device (e.g., a mobile device).

In one embodiment, the control device may calculate the reference gap value between the inner sides of the soles of both feet linked to the heel width based on the heel width value using Equation 4 below.

$${h}_{sp} = {s}_{ha} \times {h}_{4} \qquad [ Equation 4 ]$$

Here, sₕₐ may denote the heel width value, h₄ may denote a reference value of a gap range between the left and right inner sides of the soles of both feet, and hₛₚ may denote the reference gap value between the inner sides of the soles of both feet linked to the heel width. That is, the reference gap value hₛₚ between the inner sides of the soles of both feet linked to the heel width may be based on the user's heel width value sₕₐ.

Further, in one embodiment, the control device may pre-generate and store a lookup table as shown in Table 6 below based on Equation 4 described above and the user's heel width value sₕₐ described above. In this case, the control device may pre-store the lookup table and extract the reference gap value hₛₚ between the inner sides of the soles of both feet linked to the heel width based on the user's heel width value sₕₐ. Of course, the control device may generate a lookup table in more detail than Table 6 below. Further, hₛₚ 1105 to hₛₚ 1150 in Table 6 may be arbitrary numbers and may vary according to the reference value h₄ of the gap range between the left and right inner sides of the soles of both feet.

TABLE 6
Heel width sₕₐ (cm)

| | | | | | | | | | |
|---|---|---|---|---|---|---|---|---|---|
| 1 | 2 | 3 | 4 | 5 | 6 | 7 | 8 | 9 | 10 |

Reference gap value hₛₚ between inner sides of soles of both feet linked to heel width

| | | | | | | | | | |
|---|---|---|---|---|---|---|---|---|---|
| hₛₚ1105 | hₛₚ1110 | hₛₚ1115 | hₛₚ1120 | hₛₚ1125 | hₛₚ1130 | hₛₚ1135 | hₛₚ1140 | hₛₚ1145 | hₛₚ1150 |

In the example described above, when the user's heel width value is 7 cm, the reference gap value between the inner sides of the soles of both feet linked to the heel width may be hₛₚ 1135 according to Table 6.

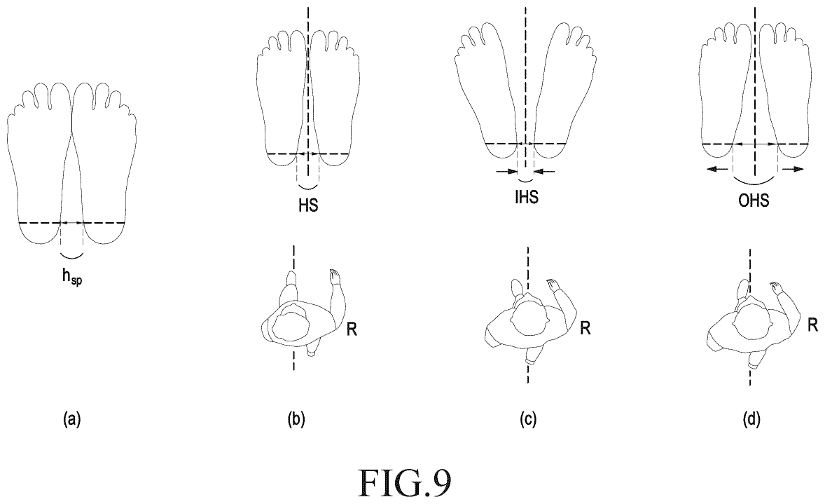

Further, in one embodiment, the control device may compare the user's measured gap value between the left and right inner sides of the soles of both feet with the reference gap value between the left and right inner sides of the soles of both feet linked to the heel width. This will be described with reference to FIG. 9.

FIG. 9 is a diagram for describing a reference gap value between left and right inner sides of soles of both feet linked to a heel width according to one embodiment.

Referring to FIG. 9, (a) illustrates a reference gap value hₛₚ between inner sides of soles of the feet linked to a heel width, and (b) to (d) illustrate a result of the comparison of the user's measured gap value between the inner sides of the soles of both feet with the reference gap value between the left and right inner sides of the soles of both feet linked to the heel width.

Specifically, (b) illustrates a state HS in which the gap value between the inner sides of the soles of the user is within a range from the reference gap value between the inner sides of the soles of both feet linked to the heel width to a predetermined value. (c) illustrates a state IHS in which the gap value between the inner sides of the soles of the user is smaller than the range from the reference gap value between the inner sides of the soles of both feet linked to the heel width to the predetermined value, and (d) illustrates a state OHS in which the gap value between the inner sides of the soles of the user is greater than the range from the reference gap value between the inner sides of the soles of both feet linked to the heel width to the predetermined value. As illustrated in FIG. 9, in the case of state HS, the user's walking posture may be appropriate. However, as in state IHS or state OHS, when the gap value between the left and right inner sides of both feet of the user is smaller or greater than the reference gap value between the inner sides of the soles of both feet linked to the heel width by a predetermined range or more, the user's walking posture may be inappropriate. Accordingly, when the gap value between the left and right inner sides of both feet of the user is smaller or greater than the reference gap value between the inner sides of the soles of both feet linked to the heel width by the predetermined range or more, the control device may provide information for adjusting the gap between the left and right inner sides of both feet of the user. For example, the control device may provide information on a difference value between the gap value between the inner sides of the soles of the user and the reference gap value between the inner sides of the soles of both feet linked to the heel width.

Further, referring to FIG. 5 again, in operation S300, the control device may calculate a reference direction value lₕₕ linked to the gap between the inner sides of the soles of both feet and the forward hand direction based on the reference gap value between the inner sides of the soles of both feet linked to the heel width and the forward hand direction value.

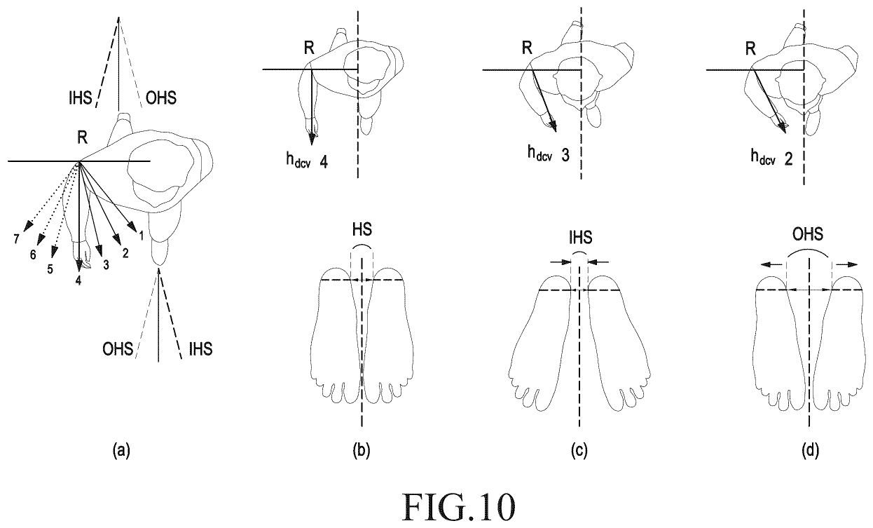

This will be described with reference to FIG. 10, and FIG. 10 is a diagram for describing a reference direction value linked to a gap between inner sides of soles of both feet and a forward hand direction.

In FIG. 10, (a) illustrates a relationship between the gap between the inner sides of the soles of both feet and the forward hand direction, and (b) to (d) illustrate a correspondence between the gap value between the left and right inner sides of the soles of both feet and the forward hand direction value. Specifically, (b) illustrates a state in which the gap value between the inner sides of the soles of both feet and the forward hand direction value correspond, and (c) and (d) illustrate states in which the gap value between the left and right inner sides of the soles of both feet and the forward hand direction value do not correspond.

As illustrated in (b) or (c), when the gap value between the left and right inner sides of the soles of both feet and the forward hand direction value do not correspond, the linked direction may be inappropriate, and thus the user's walking posture may be inappropriate. Accordingly, the control device may calculate the linked reference direction value lhw indicating the appropriate linked direction and may determine the user's walking posture based on the linked reference direction value.

In one embodiment, the control device may calculate the reference direction value linked to the gap value between the inner sides of the soles of both feet and the forward hand direction value using Equation 5 below.

[Equation 5]

lₕₕ = h_{dcv} × h₂ × sₕₐ × h₄

Here, lₕₕ may denote the reference direction value linked to the gap between the inner sides of the soles of both feet and the forward hand direction value, h_{dvc} may denote the forward hand direction calculation value, h3 may denote the reference value of the forward hand direction range of the center-of-gravity walking posture, sₕₐ may denote the heel width value, and h₄ may denote the reference value of the gap range between the left and right inner sides of the soles of both feet. According to Equation 5 above, the linked reference direction value lₕₕ may be based on the heel width value sₕₐ which is the basis of the reference gap value hₛₚ between the inner sides of the soles of both feet linked to the heel width. Further, according to Equation 5 above, the linked reference direction value lₕₕ may be based on the forward hand direction calculation value h_{dvc} which is the basis of the forward hand direction value h_{dv}.

Further, in one embodiment, the control device may pre-generate and store in a lookup table as shown in Table 7 below based on Equation 5 described above. In this case, the control device may pre-store the lookup table and extract the reference direction value linked to the gap between the inner sides of the soles of both feet and the forward hand direction value based on the lookup table. Of course, the control device may generate a lookup table in more detail than Table 7 below. For example, the lookup table in Table 5 may include the forward hand direction calculation value and the heel width value, but the present invention is not limited thereto, and the lookup table may include the forward hand direction measurement value, the forward hand direction value, and/or the gap value between the inner sides of the soles of both feet. Further, in Table 7, lₕₕ375¹ to lₕₕ875⁷ are arbitrary numbers, and may represent the reference direction values linked to the gap between the inner sides of the soles of both feet and the forward hand direction value when a reference value h3 of the forward hand direction range of the center-of-gravity walking posture and a reference value h₄ of the gap range between the left and right inner sides of the soles of both feet are predetermined to predetermined values. Accordingly, when the reference value h3 of the forward hand direction range of the center-of-gravity walking posture and the reference value of the gap range h₄ between the left and right inner sides of the soles of both feet are changed, lₕₕ375¹ to lₕₕ875⁷ in Table 7 may also be changed.

TABLE 7

| Forward hand | | | | | | |
|---|---|---|---|---|---|---|
| direction | | | | | | |
| calculation | Heel width value sₕₐ | | | | | |
| value | . . . | 3 | . . . | 5 | . . . | 7 |

| | | | | |
|---|---|---|---|---|
| 1 | . . . | lₕₕ375¹ | lₕₕ625¹ | lₕₕ875¹ |
| 2 | . . . | lₕₕ375² | lₕₕ625² | lₕₕ875² |
| 3 | . . . | lₕₕ375³ | lₕₕ625³ | lₕₕ875³ |
| 4 | . . . | lₕₕ375⁴ | lₕₕ625⁴ | lₕₕ875⁴ |
| 5 | . . . | lₕₕ375⁵ | lₕₕ625⁵ | lₕₕ875⁵ |
| 6 | . . . | lₕₕ375⁶ | lₕₕ625⁶ | lₕₕ875⁶ |
| 7 | . . . | lₕₕ375⁷ | lₕₕ625⁷ | lₕₕ875⁷ |

Further, in one embodiment, the above-described reference direction value linked to the gap between the inner sides of the soles of both feet and the forward hand direction value may be predetermined according to the user's heel width value. For example, the reference direction value linked to the gap between the inner sides of the soles of both feet and the forward hand direction value may be predetermined based on the forward hand direction value h_{dv} and the reference gap value hₛₚ between the inner sides of the soles of both feet linked to the heel width, the forward hand direction value h_{dv} may be predetermined, and the reference gap value hₛₚ between the inner sides of the soles of both feet linked to the heel width may vary according to the user's heel width value. In this way, the reference direction value linked to the gap between the inner sides of the soles of both feet and the forward hand direction value, which is predetermined according to the user's heel width value, may be the user's ideal reference direction value linked to the gap between the inner sides of the soles of both feet and the forward hand direction value.

Further, in one embodiment, the control device may calculate the user's measured linked reference direction value using Equation 6 below.

$${l}_{hh}^{′} = {h}_{sp}^{′} \times {h}_{dv}^{′} \qquad [ Equation 6 ]$$

Here, l′ₕₕ may denote the user's measured direction value, and h′ₛₚ may denote the user's measured gap value between the left and right inner sides of both feet. Further, h′_{dv} may denote the user's measured forward hand direction value.

In one embodiment, the control device may determine whether the user's measured direction value l′ₕₕ, is within a range from the reference direction value lₕₕ linked to the gap between the inner sides of the soles of both feet and the forward hand direction value to a predetermined value. When the user's measured direction value l′ₕₕ is not within the range from the reference direction value lₕₕ linked to the gap between the inner sides of the soles of both feet and the forward hand direction value to the predetermined value, the control device may provide information for correcting the user's measured direction value l′ₕₕ to the user. For example, the control device may provide information on a difference value between the user's measured direction value l′ₕₕ and the reference direction value lₕₕ linked to the gap between the inner sides of the soles of both feet and the forward hand direction value. Further, in order to correct the user's measured direction value l′ₕₕ, the control device may provide information for correcting the gap value h′ₛₚ between the left and right inner sides of the soles of both feet of the user to the heel width-linked reference gap value hₛₚ between the inner sides of the soles of both feet, and/or information for correcting the user's measured forward hand direction value h′_{dv} to the predetermined forward hand direction value h_{dv}.

Further, in operation S400, the control device may provide the walking habit correction information based on the reference stride length value laₛₜ linked to the arm swing angle and sole landing angle and/or the reference direction value lₕₕ linked to the gap between the inner sides of the soles of both feet and the forward hand direction value. In one embodiment, the control device may provide the walking habit correction information through an output unit (not illustrated) of the control device. Here, the walking habit correction information includes the correction information described in operations S200 and S300, and for example, the walking habit correction information may include information for correcting the user's arm swing angle, information for correcting the user's stride length value, information for correcting the user's sole landing angle, information for correcting the gap between the inner sides of the soles of the user's feet, information for correcting the user's forward hand direction, information for correcting the user's measured direction value, etc. Since the above-described content may be applied thereto, detailed descriptions thereof will be omitted.

Further, the control device may provide the walking habit correction information through communication with an external device (e.g., a mobile device). Further, the control device may provide the walking habit correction information to the server.

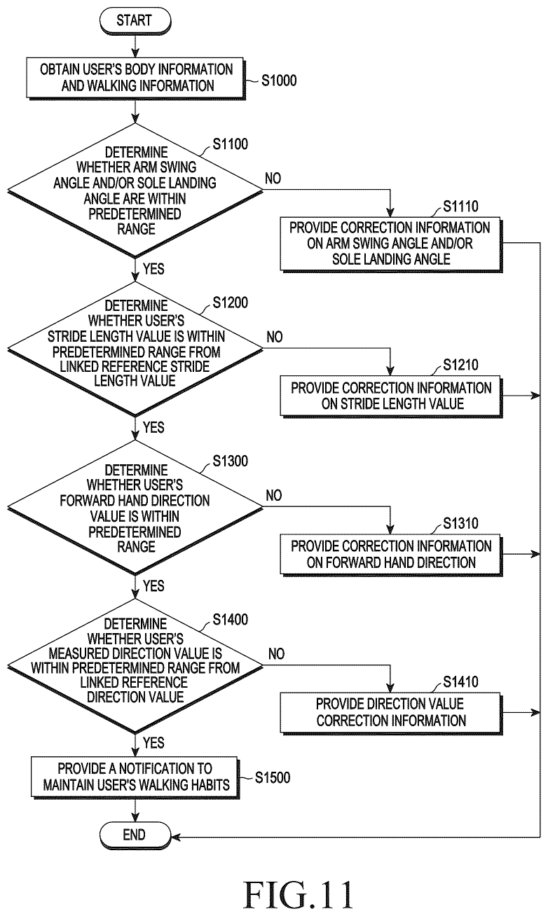

FIG. 11 is a diagram for describing a control method of a control device according to another embodiment.

Referring to FIG. 11, the control method of the control device may include operation S1000 of obtaining the user's body information and walking information, operation S1100 of determining whether an arm swing angle and/or a sole landing angle are within predetermined ranges, operation S1110 of providing correction information on the arm swing angle and/or the sole landing angle, operation S1200 of determining whether the user's stride length value is within a range from the linked reference stride length value to a predetermined value, operation S1210 of providing correction information on the stride length value, operation S1300 of determining whether the user's forward hand direction value is within a predetermined range, operation S1310 of providing correction information on the forward hand direction, operation S1400 of determining whether the user's measured direction value is within a range from the linked reference direction value to a predetermined value, operation S1410 of providing direction value correction information, and operation S1500 of providing a notification to maintain the user's walking habits. Since the above-described content may be applied to operations S1000 to S1500, detailed descriptions thereof will be omitted.

Various embodiments of the present invention may be implemented as software including instructions stored in a machine-readable storage medium (e.g., a computer-readable storage medium). The machine may include a device capable of calling the stored instructions from the storage medium and operating according to the called instructions and may include an electronic device according to the disclosed embodiments. When the instructions are executed by a processor, the processor may perform a function corresponding to the instructions using other components under the control of the processor. The instructions may include code generated or executed by a compiler or interpreter. The machine-readable storage medium may be provided in the form of a non-transitory storage medium. Here, the “non-transitory storage medium” means that the storage medium does not include a signal and is tangible and does not distinguish that data is semi-permanently or temporarily stored in the storage medium. For example, the “non-transitory storage medium” may include a buffer in which data is temporarily stored.

According to an embodiment, the methods according to various embodiments disclosed in this specification may be provided by being included in computer program products. The computer program products may be traded between sellers and buyers as commodities. The computer program products may be distributed in the form of a machine-readable storage medium (e.g., compact disc read only memory (CD-ROM)) or online through an application store (e.g., Play Store™). In the case of online distribution, at least some of the computer program products may be temporarily stored or temporarily generated in a storage medium such as a memory of a server of a manufacturer, a server of an application store, or a relay server.

As described above, while the embodiments have been described with reference to specific embodiments and drawings, various modifications and alterations may be made by those skilled in the art from the above description. For example, when the described techniques are performed in orders different from the described methods, and/or the described components such as a system, a structure, a device and a circuit are coupled or combined in a form different from the described method, or replaced or substituted with other components or equivalents, appropriate results may be achieved.

Therefore, other implementations, other embodiments and equivalents within the scope of the appended claims are included in the range of the claims to be described.

1. A control method of a control device for correcting walking habits, comprising:
obtaining a user's body information;
obtaining an arm swing angle value and a sole landing angle value while the user walks;
calculating a reference stride length value linked to the user's arm swing angle and sole landing angle based on the arm swing angle value and the sole landing angle value;
obtaining the user's heel width value;
calculating a gap value between left and right inner sides of soles of both feet based on the user's heel width value;
obtaining a forward hand direction value while the user walks;
calculating a reference direction value linked to a gap between the inner sides of the soles of both feet based on the gap value between the left and right inner sides of the soles of both feet and the forward hand direction value; and
providing walking habit correction information based on the linked reference stride length value and the linked reference direction value.

2. The control method of claim 1, wherein the user's body information includes a height value, and
the calculating of the reference stride length value linked to the user's arm swing angle and sole landing angle includes calculating a reference stride length value linked to the user's arm swing angle based on the height value and a maximum forward arm angle and a maximum rearward arm angle among the user's arm swing angles.

3. The control method of claim 2, wherein, in the calculating of the reference stride length value linked to the user's arm swing angle, the reference stride length value linked to the user's arm swing angle is calculated using a reference value of a stride length range of a center-of-gravity walking posture and a reference value of an arm swing angle range of the center-of-gravity walking posture.

4. The control method of claim 2, wherein, in the calculating of the reference stride length value linked to the user's arm swing angle and sole landing angle, the reference stride length value linked to the arm swing angle and the sole landing angle is calculated based on the reference stride length value linked to the arm swing angle and the sole landing angle value.

5. The control method of claim 4, wherein, in the calculating of the reference stride length value linked to the user's arm swing angle and sole landing angle, the reference stride length value linked to the arm swing angle and sole landing angle is calculated using a reference value of a sole landing angle range of a center-of-gravity walking posture.

6. The control method of claim 1, wherein, in the obtaining of the forward hand direction value while the user walks, a forward hand direction calculation value is obtained, and the forward hand direction value is obtained based on the forward hand direction calculation value and a reference value of a forward hand direction range of a center-of-gravity walking posture.

7. The control method of claim 6, wherein the calculating of the linked reference direction value based on the forward hand direction value and the heel width value includes calculating the gap value between the left and right inner sides of the soles of both feet based on the heel width value.

8. The control method of claim 7, wherein, in the calculating of the reference direction value linked to the gap between the inner sides of the soles of both feet, the reference direction value linked to the gap between the inner sides of the soles of both feet is calculated based on the forward hand direction value and the gap value between the left and right inner sides of the soles of both feet.

9. The control method of claim 7, wherein, in the calculating of the reference direction value linked to the gap between the inner sides of the soles of both feet, the reference direction value linked to the gap between the inner sides of the soles of both feet is calculated based on the forward hand direction calculation value, the reference value of the forward hand direction range of the center-of-gravity walking posture, and a reference value of a gap range between the left and right inner sides of the soles of both feet.

10. A non-transitory computer-readable recording medium having recorded thereon a program for performing the method of claim 1.

11. A control device for correcting walking habits, comprising:
a memory unit; and
a control unit configured to perform operations based on instructions included in the memory unit,
wherein the control unit is configured to:
obtain a user's body information,
measure an arm swing angle value and a sole landing angle value while the user walks,
calculate a reference stride length value linked to the user's arm swing angle and sole landing angle based on the arm swing angle value and the sole landing angle value;
obtain the user's heel width value,
calculate a gap value between left and right inner sides of soles of both feet based on the user's heel width value,
obtain a forward hand direction value while the user walks,
calculate a reference direction value linked to a gap between the inner sides of the soles of both feet based on the gap value between the left and right inner sides of the soles of both feet and the forward hand direction value, and
provide walking habit correction information based on the linked reference stride length value and the linked reference direction value.
